(19) United States
(12) Patent Application Publication
(10) Pub. No.: US 2005/0273047 A1
(43) Pub. Date: Dec. 8, 2005

(54) System and method for transdermal delivery of an anticoagulant

(76) Inventors: Sudeep Takhar, Gilroy, CA (US); Rama V. Padmanabhan, Los Altos, CA (US); Joseph B. Phipps, Sunnyvale, CA (US); Janardhanan A. Subramony, Santa Clara, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/144,005

(22) Filed: Jun. 1, 2005

Related U.S. Application Data
(60) Provisional application No. 60/576,905, filed on Jun. 3, 2004.

(51) Int. Cl. A61N 1/30

(52) U.S. Cl. 604/20

(57) ABSTRACT

A device for transdermally delivering an anticoagulant agent by electrotransport. Preferably, the anticoagulant comprises a benzamidine or a naphthamidine derivative. A particularly preferred benzamidine derivative is a 2-[3-[4-(4-piperidinyloxy)anilino]-1-propenyl]benzamidine derivative. The devices are configured to maintain a plasma concentration of 20-80 ng/mL or providing a flux in the range of approximately 20-40 mg/day. Suitable current densities include 0.050 and 0.10 mA/cm². Methods of the invention include delivering the anticoagulants to precisely maintain the desired plasma concentrations. The invention also comprises treating thromboembolic disease and inhibiting Factor Xa.

### CROSS REFERENCE TO RELATED U.S. APPLICATION DATA

The present application claims the benefit of provisional application 60/576,905 filed Jun. 3, 2004.

### FIELD OF THE PRESENT INVENTION

The invention relates generally to electrotransport agent delivery, and more particularly, to transdermal electrotransport agent delivery of anticoagulants. Specifically, the invention relates to a method and system of obtaining and maintaining suitable plasma concentrations of an anticoagulant, such as a benzadamine derivative, via transdermal delivery.

### BACKGROUND OF THE INVENTION

The transdermal delivery of biologically active agents or drugs offers improvements over more traditional delivery methods, such as subcutaneous injections and oral delivery. Transdermal agent delivery is an especially attractive administration route for active agent with a narrow therapeutic index, short half-life and potent activity.

Transdermal agent delivery avoids the hepatic first pass effect and gastrointestinal degradation encountered with oral active agent delivery. Transdermal agent delivery also eliminates the patient discomfort, infection risk and invasiveness associated with subcutaneous injections. In addition, transdermal agent delivery can provide more uniform concentrations of an active agent in the bloodstream of the patient over time due to the extended controlled delivery profiles of certain types of transdermal delivery devices. The term “transdermal,” as used herein, broadly encompasses the delivery of an active agent or drug through a body surface, such as the skin, mucosa, or nails of an animal.

Transdermal delivery of therapeutic agents is an important medicament administration route. As indicated, transdermal delivery bypasses gastrointestinal degradation and hepatic metabolism. Most commercial transdermal drug delivery systems (e.g., nitroglycerin, scopolamine, estradiol, testosterone skin patches) deliver active agent by passive diffusion. In the noted systems, the agent typically diffuses from a reservoir in the patch into the skin of the patient by means of the concentration gradient that exists, i.e., the agent diffuses from the high concentration in the patch reservoir to the low concentration in the patient's body. The “patch” delivery system provides slow, but controlled, delivery of the agent to a patient's blood stream.

The flux of the active agent through the patient's skin is determined by a number of factors. The factors include the agent's partition coefficient and solubility characteristics.

Unfortunately, many active agents exhibit transdermal diffusion fluxes that are too low to be therapeutically effective. This is especially true for high molecular weight agents, such as polypeptides and proteins. To enhance transdermal agent flux, a technique involving application of low levels of electric current applied through an agent reservoir in contact with a patient's body surface (e.g., skin) has been employed. This technique has been referred to as iontophoresis and, more recently, electrotransport.

As is well known in the art, electrotransport is a process by which the transdermal transport of therapeutic agents or species is achieved by using an electrical current as the driving force, i.e., by the application of an electric current to the patient through an agent-containing reservoir. As such, electrotransport is a more controllable process than passive transdermal agent delivery, since the amplitude, timing and polarity of the applied electric current is easily regulated using standard electrical components. In general, electrotransport agent flux can be several orders of magnitude greater than passive transdermal flux of the same agent.

In presently known electrotransport devices, at least two electrodes are used. Both of these electrodes are positioned in intimate electrical contact with some portion of the patient's body surface. One electrode, referred to as the active or donor electrode, is the electrode from which the therapeutic agent, agent precursor or agent is delivered into the body by electrotransport. The other electrode, referred to as the counter or return electrode, serves to close the electrical circuit through the body. In conjunction with the patient's body surface contacted by the electrodes, the circuit is completed by connection of the electrodes to a source of electrical energy, e.g., a battery.

Depending upon the electrical charge of the species to be delivered transdermally, either the anode or cathode may be the “active” or donor electrode. If, for example, the ionic substance to be delivered into the body is positively charged (i.e., a cation), then the anode will be the active electrode and the cathode will serve to complete the circuit. On the other hand, if the ionic substance to be delivered is relatively negatively charged (i.e., an anion), then the cathodic electrode will be the active electrode and the anodic electrode will be the counter electrode.

Alternatively, both the anode and the cathode may be used to deliver active agents of appropriate charge into the body. In such a case, both electrodes are considered to be active or donor electrodes. That is to say, the anodic electrode can deliver positively charged agents into the body, while the cathodic electrode can deliver negatively charged agents into the body.

Existing electrotransport devices generally require a reservoir or source of the therapeutic agent that is to be delivered into the body by electrotransport; the agent is typically in the form of a liquid solution of an ionized or ionizable species, or a precursor of such species. In some instances, the agent is formulated as a hydrogel. Examples of such reservoirs or sources include a pouch as described in Jacobsen, U.S. Pat. No. 4,250,878; a pre-formed gel body as disclosed in Drdlik, U.S. Pat. No. 4,382,529; and a glass or plastic container holding a liquid solution of the agent as disclosed in the figures of Sanderson et al., U.S. Pat. No. 4,722,726. Such agent reservoirs are electrically connected to the anode or to the cathode of the electrotransport device to provide a fixed or renewable source of one or more desired species or agents.

The term “electrotransport”, as used herein, refers generally to the electrically assisted delivery of a therapeutic agent, whether the agent to be delivered is completely charged (i.e., 100% ionized), completely uncharged, or partly charged and partly uncharged. Electromigration, electroosmosis, electroporation or any combination thereof can deliver the therapeutic agent or species thereof. Electroosmosis, in general, results from the migration of liquid solvent, in which the species is contained, as a result of the application of electromotive force to the therapeutic species reservoir. Electroporation involves the formation of transiently existing pores that occur upon applying electric current to the skin.

Of particular interest is the transdermal electrotransport delivery of agents, such as anticoagulants because of the problems encountered with more common agent administration routes, such as oral delivery. Ionically charged anticoagulants are expected to demonstrate poor permeability across the skin. However, such compounds can be effectively delivered with iontophoretic electrotransport.

One important class of anticoagulants can be characterized as Factor Xa inhibitors. Thromboembolic disease is caused by the improper functioning of the blood coagulation process. Blood clots are formed by a zymogen activation cascade of serine proteases, and the last protease of the cascade, thrombin, converts fibrinogen to fibrin, which cross-links to form blood clots. The generation of thrombin from its precursor is amplified by formation of prothrombinase complex. The protease Factor Xa has a critical function in the coagulation cascade, since it activates the generation of thrombin by the limited proteolysis of prothrombin. Thus, Factor Xa holds a central position that links the intrinsic and extrinsic activation mechanisms in the final common pathway of blood and one molecule of Factor Xa generates significant number of molecules of thrombin. Consequently, Factor Xa has emerged as an attractive target for the development of antithrombotic or anticoagulant agents, offering a potentially much more efficient means of regulation than thrombin inhibition.

A suitable class of benzamidine derivative Factor Xa inhibitors is disclosed in Japanese Patent No. 2003002832. A related reference, WO 2002089803, is directed to these types of anticoagulants and demonstrates in vitro delivery using iontophoresis. Despite the suitability of the disclosed anticoagulant agents, the references are not directed to transdermal delivery of such agents to maintain a therapeutically effective plasma concentration in vivo. The references neither suggest electrotransport conditions capable of delivering suitable doses of the agent, nor disclose alternative suitable electrotransport conditions.

A significant risk associated with anticoagulant agents is the risk of abnormal bleeding. Since an overdose of the anticoagulant can lead to bleeding due to blood thinning and an underdose of the anticoagulant will not address the condition and can lead to thrombosis, maintaining precise control of dosage is critical. These difficulties are exacerbated by characteristically low bioavailability and variable oral absorption of anticoagulants, including Factor Xa inhibitors.

Accordingly, it is an object of the invention to provide a system and method for transdermally delivering an anticoagulant agent.

It is a further object of the invention to provide a system and method for transdermally delivering an anticoagulant agent that provides precise dose control.

It is yet another object of the invention to provide a specific plasma concentration of an anticoagulant while avoiding the drawbacks associated with oral delivery.

It is another object of the present invention to provide a transdermal agent delivery and apparatus and method for maintaining a therapeutically effective plasma concentration of an anticoagulant agent.

It is another object of the present invention to provide a transdermal agent delivery apparatus and method that can easily be tailored to modify anticoagulant agent flux to generate a plasma concentration representing a therapeutically suitable level.

It is another object of the present invention to provide a transdermal agent delivery apparatus and method configured to deliver a therapeutically effective plasma concentration of an anticoagulant agent with minimal user intervention.

It is further object of the present invention to provide a transdermal anticoagulant agent delivery and apparatus that provides alternative electrotransport conditions.

### SUMMARY OF THE INVENTION

In accordance with the above objects and those that will be mentioned and will become apparent below, the invention comprises a device for transdermally delivering an anticoagulant agent by electrotransport, the device comprising a donor electrode, a donor reservoir having a source of the anticoagulant agent in a form to be delivered by electrotransport, a counter electrode, a source of electrical power and a control circuit for controlling electrotransport current, the control circuit capable of effecting electrotransport conditions configured to maintain a therapeutically desired plasma concentration of the anticoagulant agent.

Anticoagulant agents useful in the practice of the invention preferably comprise benzamidine derivatives. A particularly preferred benzamidine derivative comprises the 2-[3-[4-(4-piperidinyloxy)anilino]-1-propenyl]benzamidine derivative, referred to herein as “Compound 1”, which is shown in FIG. 3. Other suitable benzamidine derivatives comprise: N-[4-(1-acetimidoyl piperidin-4-yloxy)-3-chlorophenyl]-N-[3-(3-amidino phenyl)-2-(E)-propenyl]sulfamoyl acetic acid; N-[4-((1-acetimidoyl piperidin-4-yl)oxy)-3-carbamoyl phenyl]-N-[(E)-3-(3-amidino phenyl)-2-methyl-2-propenyl]sulfamoyl]acetic acid; N-[4-(1-aceto imidoyl piperidine-4-yloxy) phenyl]-N-[3-(3-amidino phenyl)-2-(E)-propenyl]sulfamoyl acetic acid; N-[4-(1-aceto imidoyl piperidine-4-yloxy)-3-chlorophenyl]-N-[3-(3-amidino phenyl)-2-(E)-propenyl]sulfamoyl acetic acid; N-[4-(1-aceto imidoyl piperidine-4-yloxy)-3-methylphenyl]-N-[3-(3-amidino phenyl-2-(E)-propenyl]sulfamoyl.acetic acid; N-[4-(1-aceto imidoyl piperidine-4-yloxy)-3-trifluoromethylphenyl]-N-[3-(3-amidino phenyl)-2-(E)-propenyl]sulfamoyl acetic acid; N-[4-(1-aceto imidoyl piperidine4-yloxy)-3-carbamoylphenyl]-N-[3-(3-amidino phenyl)-2-(E)-propenyl]sulfamoyl acetic acid; N-[4-(1-acelo imidoyl piperidine-4-yloxy) phenyl]-N-[3-(3-amidino phenyl)-2-fluoro-2-(Z)-propenyl]sulfamoyl acetic acid; N-[4-(1-aceto imidoyl piperidine-4-yloxy) phenyl]-N-[3-(3-amidino phenyl)-2-methyl-2-(E)-propenyl]sulfamoyl acetic acid; and N-[4-(1-aceto imidoyl piperidine-4-yloxy)-3-carbamoylphenyl]-N-[3-(3-amidino phenyl)-2-fluoro-2-(Z)-propenyl]sulfamoyl acetic acid; and pharmacologically acceptable salts thereof. Alternatively, the anticoagulant agent can also be a naphthamidine derivative.

A preferred embodiment of the invention utilizes a control circuit configured to maintain the therapeutically desired plasma concentration of the anticoagulant agent in the range of approximately 20-80 ng/mL.

In a further embodiment, the control is configured to deliver a target dose of the anticoagulant agent in the range of approximately 0.5-70 mg/day, more preferably, in the range of approximately 10-50 mg/day, even more preferably, in the range of approximately 20-40 mg/day.

In another embodiment of the invention, the control is configured to deliver a current density in the range of approximately of 0.010-0.20 mA/cm². Preferred current densities are in the range of approximately 0.050-0.10 mA/cm².

In other embodiments of the invention, the donor electrode has an area in the range of approximately 5-20 cm².

The methods and systems of the invention are capable of maintaining a therapeutically effective plasma concentration of the anticoagulant agent that is substantially equivalent to the plasma concentration maintained by intravenous infusion.

The devices of the invention can be configured to deliver direct current, alternating reverse current, or time-varying on-off electrotransport conditions.

In another aspect of the invention, the electrotransport device further comprises a blood clotting time monitor wherein the controller is configured effect the electrotransport conditions in response to a signal from the blood clotting time monitor.

The invention also comprises a method for maintaining a therapeutically effective plasma concentration of an anticoagulant agent; comprising the step of transdermally delivering by electrotransport an effective dose of said anticoagulant agent. The anticoagulant agent can comprise benzamidine or a napthamidrine derivative. Preferably, the anticoagulant agent comprises Compound 1.

More preferably, the method transdermally delivers an effective dose of Compound 1 to maintain a plasma concentration in the range of approximately 20-80 ng/mL of Compound 1.

Also preferably, the electrotransport conditions comprise applying a current density in the range of approximately 0.010-0.20 mA/cm². More preferably, the current density is in the range of approximately 0.050-0.10 mA/cm².

In the noted embodiment of the invention, the step of transdermally delivering Compound 1 comprises delivering in the range of approximately 0.5-70 mg/day, more preferably, in the range of approximately, 10-50 mg/day, even more preferably, in the range of approximately, 20-40 mg/day of Compound 1. Preferably, the noted Compound 1 delivery is achieved by applying a current density in the range of approximately 0.010-0.20 mA/cm², and more preferably, in the range of approximately 0.050-0.10 mA/cm².

The methods of the invention can comprise the use of electrotransport conditions comprising applying direct current, pulsed current, alternating reverse polarity current and time-varying on-off current.

The methods of the invention can further comprise providing a blood clotting time monitor and using a signal from the blood clotting time monitor to adjust electrotransport conditions for the step of transdermally delivering by electrotransport an effective dose of the anticoagulant agent.

The methods of the invention also comprise inhibiting Factor Xa in a patient by transdermally delivering by electrotransport a predetermined dosage of an anticoagulant agent to maintain a plasma concentration in the range of approximately 20-80 ng/mL. Preferably, the anticoagulant agent comprises Compound 1.

The methods of the invention further comprise reducing risk of thromboembolic disease in a patient by transdermally delivering by electrotransport a predetermined dosage of Compound 1 to maintain a plasma concentration in the range of approximately 20-80 ng/mL

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is an exploded perspective view of one embodiment of a device of the invention;

FIG. 2 is an illustration of a molecular structure of benzamidine moety;

FIG. 3 is an illustration of a molecular structure for a 2-[3-[4-(4-piperidinyloxy)anilino]-1-propenyl]benzamidine derivative, hereinafter referred to as “Compound 1”, that is useful in the practice of the invention;

FIGS. 4 and 5 are illustrations of molecular structures for additional benzamidine derivatives that are useful in the practice of the invention;

FIGS. 6-16 are illustrations of molecular structures for other anticoagulants that are useful in the practice of the invention;

FIG. 17 is a graph relating in vitro anticoagulant agent flux to current density;

FIG. 18 is a graph comparing in vivo and in vitro anticoagulant agent flux at various current densities;

FIG. 19 is a graph comparing plasma concentrations maintained by electrotransport in vivo delivery (ET2) to intravenous infusion; and

FIG. 20 shows useful waveforms in practicing pulsed current electrotransport conditions of the invention.

### DETAILED DESCRIPTION OF THE INVENTION

Before describing the present invention in detail, it is to be understood that this invention is not limited to particularly exemplified materials, methods or structures as such may, of course, vary. Thus, although a number of materials and methods similar or equivalent to those described herein can be used in the practice of the present invention, the preferred materials and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “an active agent” includes two or more such agents; reference to “a microprojection” includes two or more such microprojections and the like.

### Definitions

The term “transdermal”, as used herein, means the delivery of an agent into and/or through the skin for local or systemic therapy.

The term “transdermal flux”, as used herein, means the rate of transdermal delivery.

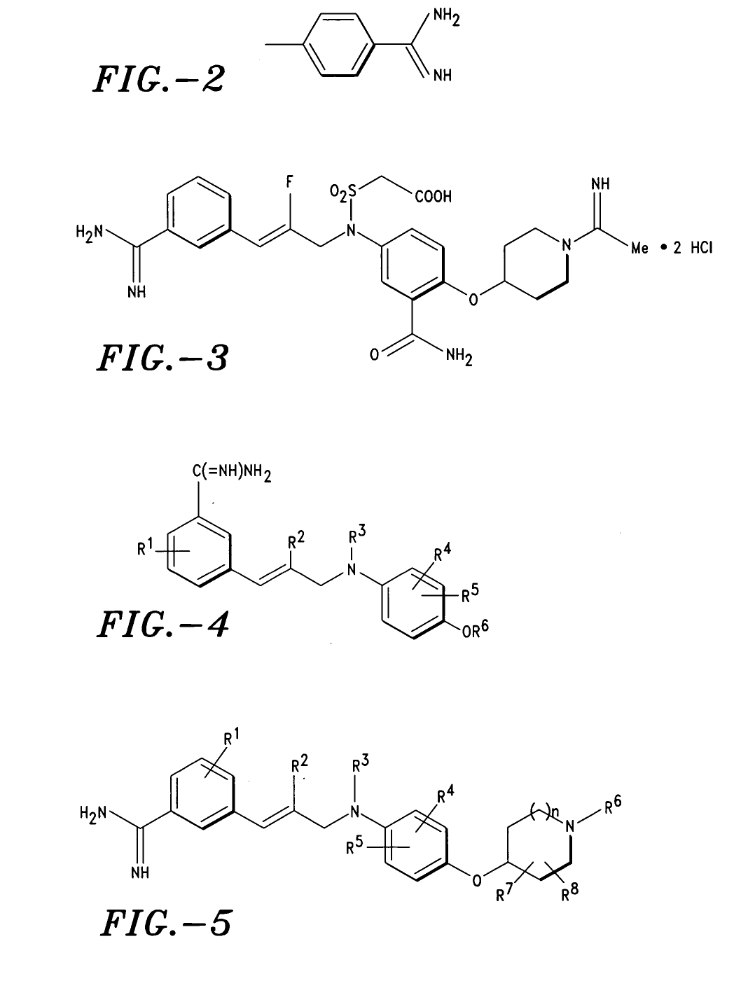

The term “anticoagulant agent”, as used herein, may be used interchangeably and synonymously with the term “antithrombotic agents.” These terms apply to any compositions that inhibit or compete with coagulation processes. A preferred class of anticoagulant agents comprise benzamidine derivatives that inhibit Factor Xa. A preferred 2-[3-[4-(4-piperidinyloxy)anilino]-1-propenyl]benzamidine derivative, termed “Compound 1”, is particularly suitable for the practice of the invention (see FIG. 3). As is well known in the art, the noted benzamidine derivatives comprise synthetic cationic agents having relatively low molecular weight, such as about 500 to 600 Daltons. Another class of suitable anticoagulant agents comprises naphthamidine derivatives.

The noted anticoagulant agents can also be in various forms, such as free bases, acids, charged or uncharged molecules, components of molecular complexes or non-irritating, pharmacologically acceptable salts. Further, simple derivatives of the active agents (such as ethers, esters, amides, etc.), that are easily hydrolyzed at body pH, enzymes, etc., can be employed.

It is to be understood that more than one anticoagulant agent may be incorporated into the agent's source or reservoir of this invention, and that the use of the term “agent” in no way excludes the use of two or more such active agents.

The term “biologically effective amount” or “biologically effective rate” shall be used when the biologically active agent is a pharmaceutically active agent and refers to the amount or rate of the pharmacologically active agent needed to effect the desired therapeutic, often beneficial, result. In preferred embodiments, this comprises a therapeutically significant diminution of risk of thrombosis or other thromboembolic disease or condition. The amount of active agent employed in the agent formulations of the invention will be that amount necessary to deliver a therapeutically effective amount of the anticoagulant agent to achieve the desired therapeutic result. In practice, this will vary widely depending upon the particular pharmacologically active agent being delivered, the site of delivery, the severity of the condition being treated, the desired therapeutic effect and the dissolution and release kinetics for delivery of the agent from the coating into skin tissues.

The term “electrotransport,” as used herein, refers generally to the delivery or extraction of a therapeutic agent (charged, uncharged, or mixtures thereof) through a body surface (such as skin, mucous membrane, or nails) wherein the delivery or extraction is at least partially induced or aided by the application of an electric potential. As is well known in the art, a widely used electrotransport process, electromigration (also called iontophoresis), involves the electrically induced transport of charged ions (e.g., agent ions) through a body surface. Another type of electrotransport, called electroosmosis, involves the trans-body surface (e.g., transdermal) flow of a liquid under the influence of the applied electric field.

One widely used electrotransport process, iontophoresis, involves the electrically induced transport of charged ions. Electroosmosis, another type of electrotransport process involved in the transdermal transport of uncharged or neutrally charged molecules (e.g., transdermal sampling of glucose), involves the movement of a solvent with the agent through a membrane under the influence of an electric field. The term “electroporation”, as used herein, generally recognizes that exposing cells to strong electric fields for brief periods of time can temporarily destabilize the biological membranes. This effect may also be referred to as “electropermeabilization.”

In many instances, more than one of the noted processes may be occurring simultaneously to different extents. Accordingly, the term “electrotransport” is given herein its broadest possible interpretation, to include the electrically induced or enhanced transport of at least one charged or uncharged agent, or mixtures thereof, regardless of the specific mechanism(s) by which the agent is actually being transported.

As indicated above, the present invention comprises an apparatus and system for transdermally delivering an anticoagulant agent to a patient. The system generally includes an active electrode and a donor electrode and electric circuitry for supplying electrical signals to the electrodes. Further, a source of the anticoagulant agent is provided adjacent at least one of the electrodes.

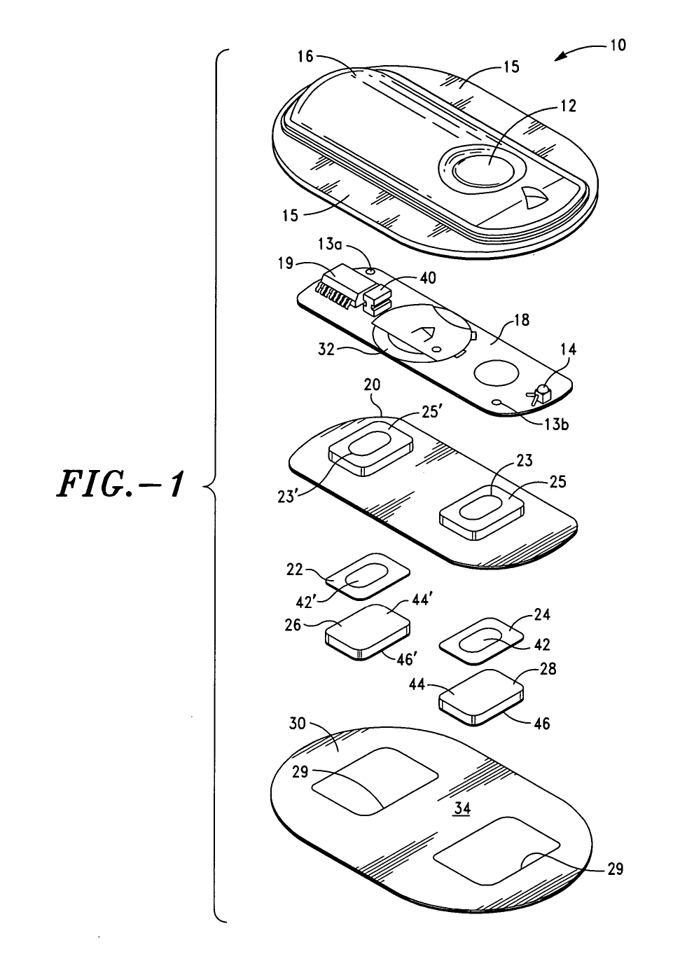

Reference is now made to FIG. 1, which depicts an exemplary electrotransport device that can be used in accordance with the present invention. FIG. 1 shows a perspective exploded view of an electrotransport device 10 having an activation switch in the form of a push button switch 12 and a display in the form of a light emitting diode (LED) 14. Device 10 comprises an upper housing 16, a circuit board assembly 18, a lower housing 20, anode electrode 22, cathode electrode 24, anode reservoir 26, cathode reservoir 28 and skin-compatible adhesive 30. Upper housing 16 has lateral wings 15 that assist in holding device 10 on a patient's skin. Upper housing 16 is preferably composed of an injection moldable elastomer (e.g., ethylene vinyl acetate). Printed circuit board assembly 18 comprises an integrated circuit 19 coupled to discrete electrical components 40 and battery 32. Circuit board assembly 18 is attached to housing 16 by posts (not shown in FIG. 1) passing through openings 13a and 13b, the ends of the posts being heated/melted in order to heat stake the circuit board assembly 18 to the housing 16. Lower housing 20 is attached to the upper housing 16 by means of adhesive 30, the upper surface 34 of adhesive 30 being adhered to both lower housing 20 and upper housing 16 including the bottom surfaces of wings 15.

Shown (partially) on the underside of circuit board assembly 18 is a battery 32, preferably, a button cell battery and most preferably a lithium cell. Other types of batteries may also be employed to power device 10.

The circuit outputs (not shown in FIG. 1) of the circuit board assembly 18 make electrical contact with the electrodes 24 and 22 through openings 23, 23′ in the depressions 25, 25′ formed in lower housing, by means of electrically conductive adhesive strips 42, 42′. Electrodes 22 and 24, in turn, are in direct mechanical and electrical contact with the top sides 44′, 44 of reservoirs 26 and 28. The bottom sides 46′, 46 of reservoirs 26, 28 contact the patient's skin through the openings 29′, 29 in adhesive 30. Upon depression of push button switch 12, the electronic circuitry on circuit board assembly 18 delivers a predetermined DC current to the electrodes/reservoirs 22, 26 and 24, 28 for a delivery interval of predetermined length, e.g., about 10 minutes. Preferably, the device transmits to the user a visual and/or audible confirmation of the onset of the agent delivery, or bolus, interval by means of LED 14 becoming lit and/or an audible sound signal from, e.g., a “beeper.”

Anodic electrode 22 and/or cathodic electrode 24 may be preferably comprised of silver and/or silver chloride, or any suitable electrically conductive material and both reservoirs 26 and 28 are preferably comprised of polymeric materials, as described below. Electrodes 22, 24 and reservoirs 26, 28 are retained by lower housing 20. For anionic biologically active agents, the cathodic reservoir 28 is the “donor” reservoir, which contains the active agent, and the anodic reservoir 26 contains a biocompatible formulation. One having ordinary skill in the art will readily recognize that with cationic biologically active agents, the reservoirs 26, 28 are reversed.

The agent reservoir 26 and return reservoir 28 of the iontophoretic delivery device 10 must be placed in an agent transmitting relation with the patient so as to iontophoretically deliver the agent. Usually this means the device is placed in intimate contact with the patient's skin. Various sites on the human body may be selected depending upon the physician's or the patient's preference, the agent delivery regimen or other factors such as cosmetic.

The donor and counter electrodes 22 and 24 are positioned adjacent to the donor reservoir 26 and the counter agent reservoir 28, respectively. The donor reservoir 26 contains the agent to be delivered, while the counter reservoir 28 typically contains a biocompatible electrolytic salt. The donor reservoir 26 and optional counter agent reservoir 28 may be any material adapted to absorb and hold a sufficient quantity of liquid therein in order to permit transport of agent therethrough by electrotransport. For example, gauzes, pads or sponges composed of cotton or other absorbent fabric, both natural and synthetic, may be used.

More preferably, the matrices of the reservoirs 26 and 28 are composed, at least in part, of a hydrophilic polymer material. Hydrophilic polymers are preferred because water is the preferred ion transport medium, and hydrophilic polymers have a relatively high equilibrium water content. Most preferably, the matrices of the reservoirs 26 and 28 are solid polymer matrices composed, at least in part, of insoluble hydrophilic polymer. Insoluble hydrophilic polymer matrices are preferred for structural reasons over soluble hydrophilic polymers.

The matrices can be cross-linked with the agent components in place such as a silastic matrix, or the polymers can be prefabricated and sorbed with the components from solutions as is the case with cellulose, woven fiber pads and sponges. The agent reservoirs 26 and 28 can alternately be a gel matrix structure, formed similarly to the polymeric matrix structure, wherein the gel is formed of a hydrophilic polymer which is swellable or soluble in water. Such polymers may be blended with the components in any ratio, but preferably represent from a few to about 50 wt % of the reservoir. The polymers may be linear or cross-linked. Suitable hydrophilic polymers include co-polyesters such as HYTREL (DuPont De Nemours & Co., Wilmington, Del.), polyvinylpyrrolidones, polyvinyl alcohol, polyethylene oxides such as POLYOX (Union Carbide Corp.), CARBOPOL (BF Goodrich of Akron, Ohio), blends of polyoxyethylene or polyethylene glycols with polyacrylic acid such as POLYOX blended with CARBOPOL, polyacrylamide, KLUCEL, cross-linked dextran such as SEPHADEX (Pharmacia Fine Chemicals, AB, Uppsala, Sweden), WATER LOCK (Grain Processing Corp., Muscatine, Iowa) which is a starch-graft-poly(sodium acrylate-co-acrylamide) polymer, cellulose derivatives such as hydroxyethyl cellulose, hydroxypropyl-methylcellulose, low-substituted hydroxypropylcellulose, and cross-linked Na-carboxymethylcellulose such as Ac-Di-Sol (FMC Corp., Philadelphia, Pa.), hydrogels such as polyhydroxylethyl methacrylate (National Patent Development Corp.), natural gums, chitosan, pectin, starch, guar gum, locust bean gum, and the like, along with blends thereof. The noted list is merely exemplary of the materials suited for use in this invention. Other suitable hydrophilic polymers can be found in Scott, J. R., & Roff, W. J., Handbook of Common Polymers, CRC Press (1971), the pertinent portions of which being hereby incorporated by reference.

The matrices of the reservoirs 26 and 28 may optionally contain a hydrophobic polymer for enhanced structural rigidity. Preferably, the hydrophobic polymer is heat fusible, in order to improve the lamination of the reservoirs to adjacent components. Suitable hydrophobic polymers for use in the reservoir matrices include, but are not limited to, polyisobutylenes, polyethylene, polypropylene, polyisoprenes and polyalkenes, rubbers, copolymers such as KRATON, polyvinylacetate, ethylene vinyl acetate copolymers, polyamides such as nylons, polyurethanes, polyvinylchloride, acrylic or methacrylic resins such as polymers of esters of acrylic or methacrylic acid with alcohols such as n-butanol, 1-methyl pentanol, 2-methyl pentanol, 3-methyl pentanol, 2-ethyl butanol, isooctanol, n-decanol, alone or copolymerized with ethylenically unsaturated monomers such as acrylic acid, methacrylic acid, acrylamide, methacrylamide, N-alkoxymethyl acrylamides, N-alkoxymethyl methacrylamides, N-tert-butylacrylamide, itaconic acid, N-branched alkyl maleamic acids wherein the alkyl group has 10-24 carbon atoms, glycol diacrylates, and blends thereof. Most of the above-mentioned hydrophobic polymers are heat fusible.

The reservoir matrices can be a polymeric matrix structure formed by blending the desired agent, electrolyte, or other component(s), with an inert polymer by such processes as melt blending, solvent casting, or extrusion.

According to the invention, the counter reservoir 28 may contain any one or more of the following electrolytes: alkali metal salts such as NaCl; alkaline earth metal salts such as chlorides, sulfates, nitrates, carbonates, and phosphates; organic salts such as ascorbates, citrates, and acetates; electrolytes containing redox species such as copper ions, iron ions, quinone, hydroquinone, silver ions, and 10 ions; and other biocompatible salts and buffers. Sodium chloride is the preferred electrolytic salt for the counter reservoir 28.

In addition to the agent to be delivered and electrolyte, the reservoirs 26 and 28 can also contain other conventional materials such as dyes, pigments, inert fillers, and the like.

Examples of suitable metals for electrodes include, but is not limited to, silver, zinc, silver chloride, aluminum, platinum, stainless steel, gold, and titanium. Most preferably, the anodic electrode is comprised of silver, while the cathodic electrode is comprised of silver chloride. Silver is preferred as an anode over other metals because of its relatively low toxicity to humans. Silver chloride is preferred as a cathode material because the reduction of silver chloride produces chloride ions that are endogenous to the human body.

Generally, the combined skin-contacting area of electrode assemblies are in the range of approximately 1-200 cm², but typically will be in the range of approximately 5-50 cm².

In a preferred embodiment, the push button switch 12, the electronic circuitry on circuit board assembly 18 and the battery 32 are adhesively “sealed” between upper housing 16 and lower housing 20. Upper housing 16 is preferably composed of rubber or other elastomeric material. Lower housing 20 is preferably composed of a plastic or elastomeric sheet material (e.g., polyethylene) which can be easily molded to form depressions 25, 25′ and cut to form openings 23, 23′. The assembled device 10 is preferably water resistant (i.e., splash proof) and is most preferably waterproof.

The system has a low profile that easily conforms to the body thereby allowing freedom of movement at, and around, the wearing site.

The anode/agent reservoir 26 and the cathode/salt reservoir 28 are located on the skin-contacting side of device 10 and are sufficiently separated to prevent accidental electrical shorting during normal handling and use.

In a preferred embodiment, the device 10 adheres to the patient's body surface by means of a peripheral adhesive 30 that has upper side 34 and body-contacting side (not shown). The adhesive side 36 has adhesive properties which assures that the device 10 remains in place on the body during normal user activity, and yet permits reasonable removal after the predetermined (e.g., 24-hour) wear period. Upper adhesive side 34 adheres to lower housing 20 and retains the electrodes and agent reservoirs within housing depressions 25, 25′ as well as retains lower housing 20 attached to upper housing 16.

The push button switch 12 is preferably located on the top side of device 10 and is easily actuated through clothing. Upon switch activation, a first electric signal configured to facilitate transdermal transport as described herein or a second electric signal configured to facilitate intracellular transport as also described herein can be initiated. Alternatively, the operation can be automated.

In one embodiment of electrotransport, an audible alarm signals the start of agent delivery, at which time the circuit supplies a predetermined level of DC current to the electrodes/reservoirs for a predetermined delivery interval. The LED 14 remains “on” throughout the delivery interval indicating that the device 10 is in an active agent delivery mode. The battery preferably has sufficient capacity to continuously power the device 10 at the predetermined level of DC current for the entire (e.g., 24 hour) wearing period.

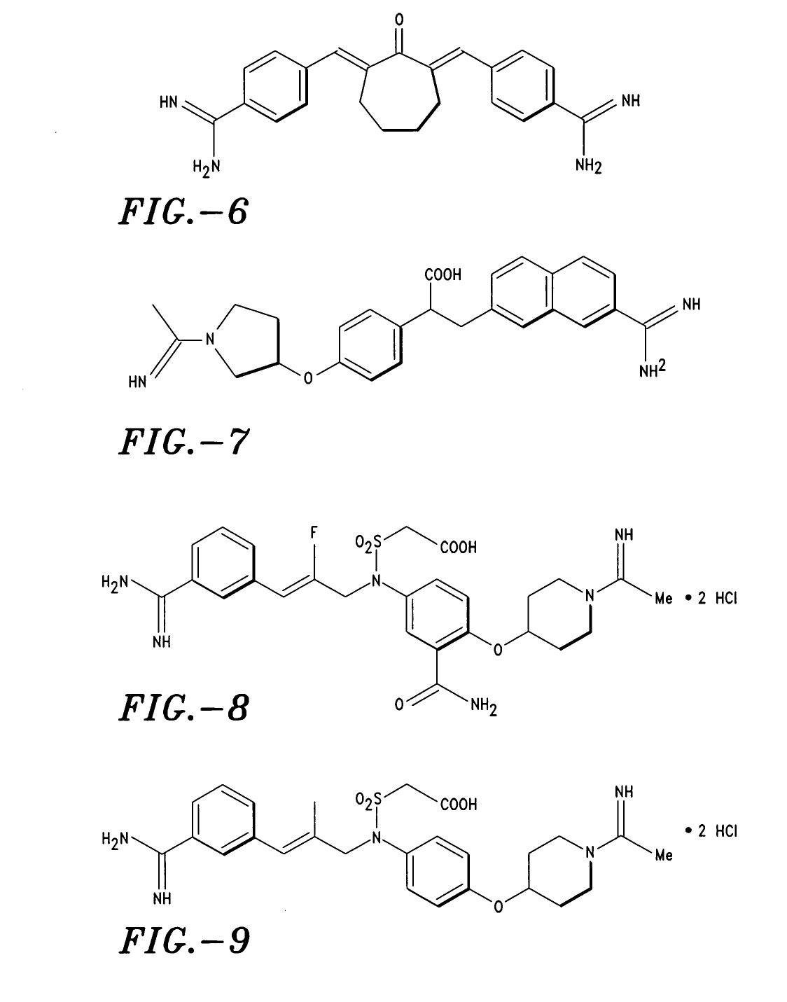

As discussed above, preferred agents for transdermal delivery using the systems and methods of the invention comprise anticoagulants or antithrombotics that inhibit or compete with coagulation processes. A preferred class of agents is benzamidine derivatives that inhibit Factor Xa. Suitable compounds exhibit two basic benzamidine moities, shown in FIG. 6, symmetrically situated and separated by a spacer containing other moieties of appropriate length.

Referring now to FIG. 3, there is shown a representative synthetic derivative, the 2-[3-[4-(4-piperidinyloxy)anilino]-1-propenyl]benzamidine derivative, referred to herein as “Compound 1.” Compound 1 is a synthetic cationic agent having a molecular weight in the range of 500 to 600 Daltons.

In other embodiments of the invention, similar benzamidine derivatives or their pharmacologically acceptable salts generally represented by the formula shown in FIG. 4, can be employed, wherein, R<1> is H, a halogen atom, an alkyl group or OH; R<2> is H, a halogen atom or an alkyl group; R<3> is H, an alkyl group which may have a substituent, an aralkyl group, an alkanoyl group which may have a substituent or an alkylsulfonyl group which may have a substituent; R<4> and R<5> are each independently H, a halogen atom, an alkyl group which may have substituent, an alkoxy group, carboxyl group, an alkoxycarbonyl group or a carbamoyl group which may have substituent; and R<6> is a substituted pyrrolidine or a substituted piperidine.

In a preferred embodiment, R<1> is H, a halogen atom, a C1-6 alkyl group or OH; R<2> is H, a halogen atom or a C1-6 alkyl group; R<3> is H, a C1-6 alkyl group which may be substituted with OH, CO2H, or C1-6 alkoxy carbonyl, (CH2)nCO(CH2)mCO2R7 (wherein R7 is a C1-6 alkyl and m and n each independently are 1-6), C7-15 aralkyl, C1-6 alkanoyl, C2-6 hydroxyalkanoyl, C1-6 alkylsulfonyl, C1-6 alkoxy-crbonyl, carboxyl-C1-6 alkylsulfonyl; R<4> and R<5> are each independently H, a halogen atom, a C1-6 (halogen)alkyl, C1-6 alkoxy, CO2H, C1-6 alkoxy-carbonyl, CONH2, C1-6 alkyl-carbamoyl, and di(C1-6 alkyl)carbamoyl; and R<6> is 1-acetimidoylpyrrolidin-3-yl or 1-acetimidoylpiperidin-4-yl. A particularly preferred compound comprises N-[4-(1-acetimidoyl piperidin-4-yloxy)-3-chlorophenyl]-N-[3-(3-amidino phenyl)-2-(E)-propenyl]sulfamoylacetic acid dihydrochloride (see FIG. 5).

According to the invention, other suitable benzamidine derivatives (and their pharmacologically acceptable salts) that are similar to Compound 1 and have the general formula shown in FIG. 5 can be employed, wherein R<1> represents hydrogen, a halogen, an alkyl, or hydroxy; R<2> represents hydrogen, a halogen, or an alkyl; R<3> represents hydrogen, an optionally substituted alkyl, an optionally substituted acyl, or an optionally substituted alkylsulfonyl; R<4> and R<5> are the same or different and each represents hydrogen, halogen, an optionally substituted alkyl, alkoxy, carboxyl, alkoxycarbonyl, or an optionally substituted carbamoyl; R<6> represents hydrogen, an optionally substituted alkyl, an optionally substituted acyl, carbamoyl, alkylsulfonyl, aryl, etc.; R<7> and R<8> are the same or different and each represents hydrogen, an alkyl or the like and n is 0, 1, or 2) or a pharmacologically acceptable salt of the derivative. In a preferred embodiment, R<1> represents hydrogen, a halogen, an alkyl, or hydroxy; R<2> represents hydrogen, a halogen, or a C1-6 alkyl; R<3> represents hydrogen, C1-6 alkyl, C1-6 hydroxyalkyl, C2-7 carboxyalkyl, C3-13 alkoxycarbonylalkyl, C7-16 aralkyl, C2-7 aliphatic acyl, C2-7 hydroxy-aliphatic acyl, C1-6 alkylsulfonyl, C30-13 alkoxycarbonylalkylsulfonyl, C2-7 carboxyalkylsulfonyl, or C3-8 carboxyalkylcarbonyl; R<4> and R<5> are each independently hydrogen, a halogen, C1-6 alkyl, C1-6 haloalkyl, C1-6 alkoxy, CO2H, C2-7 alkoxycarbonyl, CONH2, C2-7 monoalkyl or C3-13 dialkylcarbamoyl; R<6> represents hydrogen, C1-6 alkyl, C3-8 cycloalkyl, C7-16 aralkyl, heterocyclyl-C1-6 alkyl, C2-7 carboxyalkyl, C3-13 alkoxycarbonylalkyl, C2-7 aliphatic acyl, C7-11 armatic acyl, CONH2, C1-6 alkylsulfonyl, C6-10 aryl, heterocyclyl, formimidoyl, C2-7 1-iminoalkyl, C2-7 N-alkylformimidoyl, or C7-11 iminioarylmethyl; and R<7> and R<8> are each independently hydrogen, C1-6 alkyl; or R<6>, R<7> and R<8> together represent C2-5 alkylene; and n is 0, 1, or 2.

In a preferred embodiment, the above composition comprises [N-[4-((1-acetimidoyl piperidin-4-yl)oxy)-3-carbamoyl phenyl]-N-[(E)-3-(3-amidino phenyl)-2-methyl-2-propenyl]sulfamoyl]acetic acid dihydrochloride. It may be prepared by adding 0.39 g Et acetimidate hydrochloride and 0.87 mL Et3N to a solution of [N-[(E)-3-(3-amidinophenyl)-2-methyl-2-propenyl]-N-[3-carbamoyl-4(piperidin-4-yloxy)phenyl]sulfamoyl]acetic acid Et ester in 20 mL ethanol and stirred at room temperature for 6 hours to give 75% [N-[4-((1-acetimidoylpiperidin-4-yl)oxy)-3-carbamoyl-N-[(E)-3-(3-amidinophenyl)-2-methyl-2-propenyl]phenyl]sulfamoyl]acetic acid Et ester dihydrochloride which (0.64 g) is dissolved in 20 mL 3 N aqueous HCL and heated at 80° C. for 2 hours.

Other suitable benzamidine derivatives or pharmacologically acceptable salt thereof include N-[4-(1-.aceto imidoyl piperidine-4-yloxy) phenyl]-N-[3-(3-amidino phenyl)-2-(E)-propenyl]sulfamoyl acetic acid; N-[4-(1-aceto imidoyl piperidine-4-yloxy)-3-methylphenyl]-N-[3-(3-amidino phenyl-2-(E)-propenyl]sulfamoyl acetic acid; N-[4-(1-aceto imidoyl piperidine-4-yloxy)-3-trifluoromethylphenyl]-N-[3-(3-amidino phenyl)-2-(E)-propenyl]sulfamoyl acetic acid; N-[4-(1-aceto imidoyl piperidine-4-yloxy)-3-carbamoylphenyl]-N-[3-(3-amidino phenyl)-2-(E)-propenyl]sulfamoyl acetic acid; N-[4-(1-acelo imidoyl piperidine-4-yloxy) phenyl]-N-[3-(3-amidino phenyl)-2-fluoro-2-(Z)-propenyl]sulfamoyl acetic acid; N-[4-(1-aceto imidoyl piperidine-4-yloxy) phenyl]-N-[3-(3-amidino phenyl)-2-methyl-2-(E)-propenyl]sulfamoyl acetic acid; and N-[4-(1-aceto imidoyl piperidine-4-yloxy)-3-carbamoylphenyl]-N-[3-(3-amidino phenyl)-2-fluoro-2-(Z)-propenyl]sulfamoyl acetic acid.

There are a number of other Factor Xa inhibitors useful for practicing the invention. For example, the compound shown in FIG. 6 was developed by Pentapharm Ltd, of Basel, Switzerland. The noted compound, a benzamidrine derivative Factor Xa inhibitor, was the first non-peptidic compound shown to have a high affinity for Factor Xa.

Another example is the compound shown in FIG. 7, which is a Factor Xa inhibitor developed by Daiichi Pharmaceuticals. As illustrated in FIG. 7, the noted compound contains a naphthamidine group rather than a benzamidine group. The compound also has a free carboxylic acid group that has been identified as being essential for Factor Xa selectivity. It has been found that the carboxylic acid enhances Factor Xa selectivity by about 100, fold as compared to the corresponding methyl ester.

There are some significant similarities between the DX-9065a compound and Compound 1. Specifically, they both have carboxylic acid group that appears to enhance the activity of the compounds. Both compounds also have an oxy bridge to connect the functional groups and an acetamidol group.

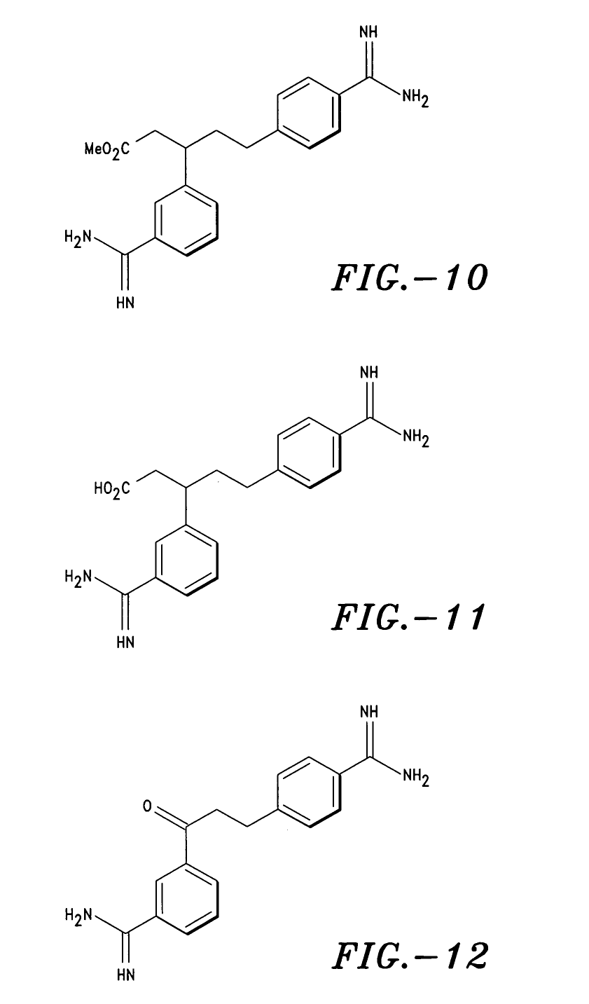

Additional suitable Factor Xa inhibitors include “Compound 2”, shown in FIG. 8 and “Compound 3”, shown in FIG. 9. Another suitable Factor Xa inhibitor comprises YM-60828, which comprises [N-[4-[(1-acetimidoyl-4-piperidyl)-oxy)phenyl)-N-(7-amidino-2-naphthyl)methyl]sulfamoyl]-acetic acid and is structurally similar to DX-9065a, is illustrated and discussed in detail in Sato, et al., Antithrombotic Effects of YM-60828, A Newly Synthesized Factor Xa Inhibitor, in Rat Thrombosis Models and its Effects on Bleeding Time, Br. J. Pharmacol, vol. 123:92-6 (1998), the relevant sections of which are incorporated by reference herein.

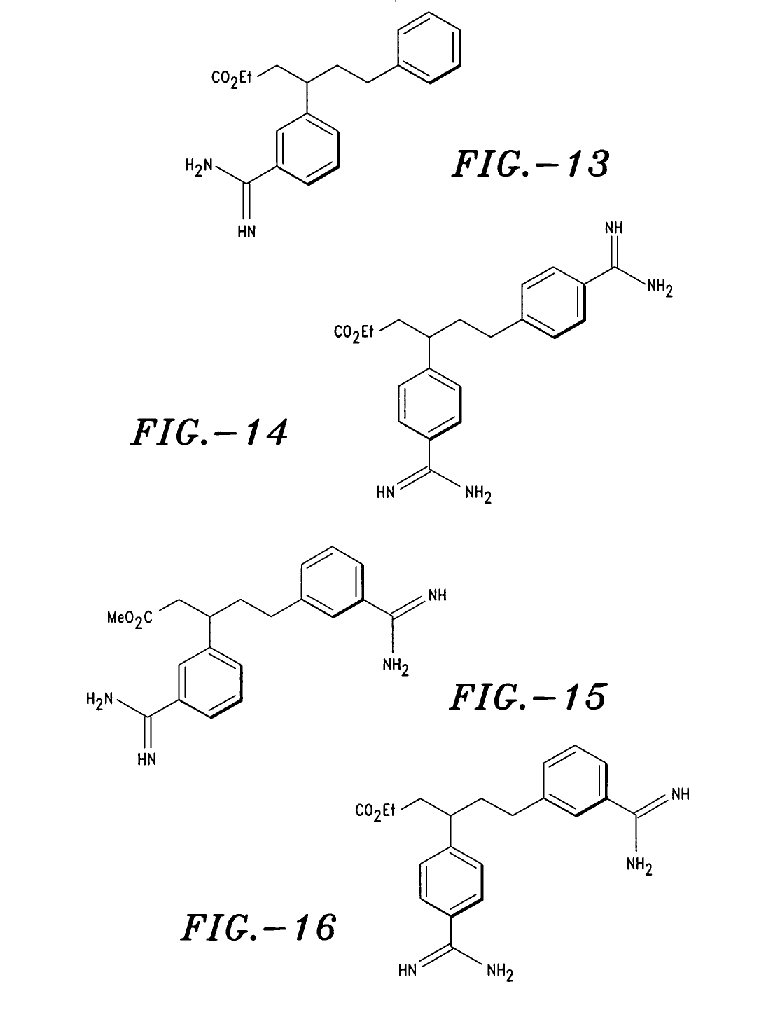

Yet other examples of benzamidine derivatives are shown in FIGS. 10-16, which are a Dupont-Merck synthetic series of bisbenzamidine derivatives having a carboxylic acid group.

As stated, electrotransport embodiments of the invention employ at least two electrodes that are in electrical contact with some portion of the skin, nails, mucous membrane, or other surface of the body. One electrode, commonly referred to as the “donor” electrode, is the electrode from which the therapeutic agent is delivered into the body. The other electrode, typically termed the “counter” electrode, serves to close the electrical circuit through the body.

Furthermore, electrotransport delivery systems generally require at least one reservoir or source, of the therapeutic agent to be delivered to the body. Examples of such donor reservoirs include a pouch or cavity, a porous sponge or pad, and a hydrophilic polymer or a gel matrix. Such donor reservoirs are electrically connected to, and positioned between, the anode or cathode and the body surface, to provide a fixed or renewable source of one or more therapeutic agents.

As indicated, electrotransport devices are powered by an electrical power source, such as one or more batteries. Typically, at any one time, one pole of the power source is electrically connected to the donor electrode, while the opposite pole is electrically connected to the counter electrode. Since it has been shown that the rate of electrotransport agent delivery is approximately proportional to the electric current applied by the device, many electrotransport devices typically have an electrical controller that controls the voltage and/or current applied through the electrodes, thereby regulating the rate of agent delivery. These control circuits use a variety of electrical components to control the electrical signal, i.e., the amplitude, polarity, timing, waveform shape, etc., of the electric current and/or voltage, supplied by the power source. U.S. Pat. No. 5,047,007 to McNichols, et al., which is hereby incorporated by reference in its entirety, discloses several suitable parameters and characteristics.

In particular, the use of a direct current applied across the two electrodes represents the most straightforward application of the invention. The use of a constant direct current signal typically provides a very linear relation ship between the applied current density and the flux of the anticoagulant agent.

As discussed above, it is critical to maintain accurate dosing of the anticoagulant agent. Under-dosing does not provide the necessary inhibition of the coagulation pathways, increasing the risk of thrombosis or other thromboembolic condition. In contrast, overdosing increases the risk of unwanted or uncontrollable bleeding due to the interference with the coagulation process.

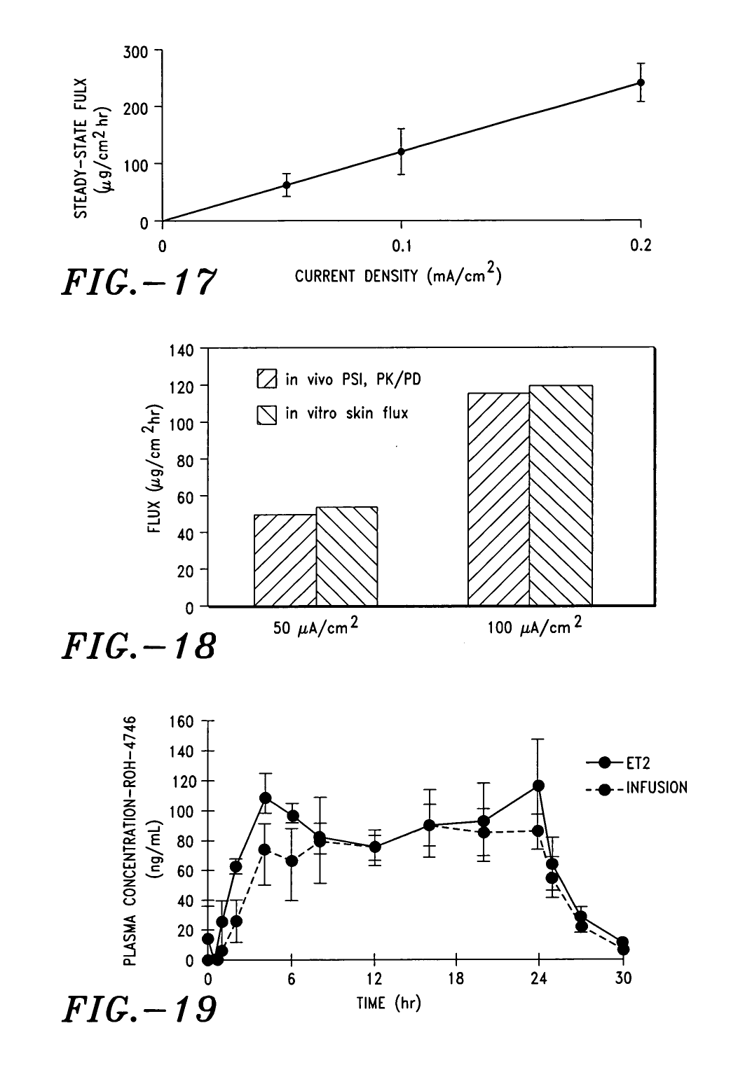

Suitable anticoagulant flux rates can be achieved by selecting appropriate electrotransport conditions. As shown in FIG. 17, the inventive electrotransport systems and methods of the invention provide an accurate correlation between the applied current and steady state agent flux in vitro. This data was obtained using the transdermal delivery of Compound 1 with a silver donor electrode at the anode and a silver chloride counter electrode at the cathode. As demonstrated, there is a linear correlation between the magnitude of applied current to the steady state flux with an in vitro transport efficiency of about 1.1 mg/mAhr. The tests were performed on heat separated human epidermis.

With regard to Compound 1, a useful therapeutic range of plasma concentration is in the range of approximately 20-80 ng/mL. Given the linear relationship between applied current and agent flux, one of skill in the art can select appropriate electrotransport conditions to achieve therapeutic dosing. Table 1 provides a range of electrotransport conditions suitable to provide a therapeutic dose of Compound 1, at an operating current density of 0.05 mA/cm². The inventive transdermal electrotransport avoids the drawbacks associated with oral delivery, such as poor oral bioavailability, variable oral absorption, gastrointestinal degradation or hepatic first pass effects.

TABLE 1

| Plasma Concentration | Infusion Rate | Current | Area |
|---|---|---|---|
| (ng/mL) | mg/hour | (mA) | (cm²) |
| 80 | 1 | 1 | 20 |
| 40 | 0.5 | 0.5 | 10 |
| 20 | 0.25 | 0.25 | 5 |

The in vitro correlation observed in FIG. 17 is further supported by the extraction of residual Compound 1 in in vivo studies comprising Primary Skin Irritation (PSI) in hairless guinea pigs and Pharmaco-Kinetic (PK) in swine. The data is shown in Table 2.

TABLE 2

| | Estimated Flux |
|---|---|
| In Vivo Study | (microg/cm² hr) |
| PSI Study in hairless guinea pigs | 60 |
| at 0.050 mA/cm² | |
| PK swine un-buffered at 0.1 mA/cm² | 117 |
| PK swine buffered at 0.050 mA/cm² | 66 |
| PK swine buffered at 0.1 mA/cm² | 135 |

Referring now to FIG. 18, further evidence of in vitro and in vivo correlation is shown. Specifically, data from in vivo experiments are comparable to in vitro electrotransport experiments. As can be seen, at the lower 0.050 mA/cm² current density, a comparison of in vivo PSI and in vitro skin flux of a buffered Compound 1 formulation yield similar steady-state flux profiles. Similar results are shown to be obtained in the higher 0.10 mA/cm² current density in vivo PK/PD and the in vitro skin flux-unbuffered formulation data.

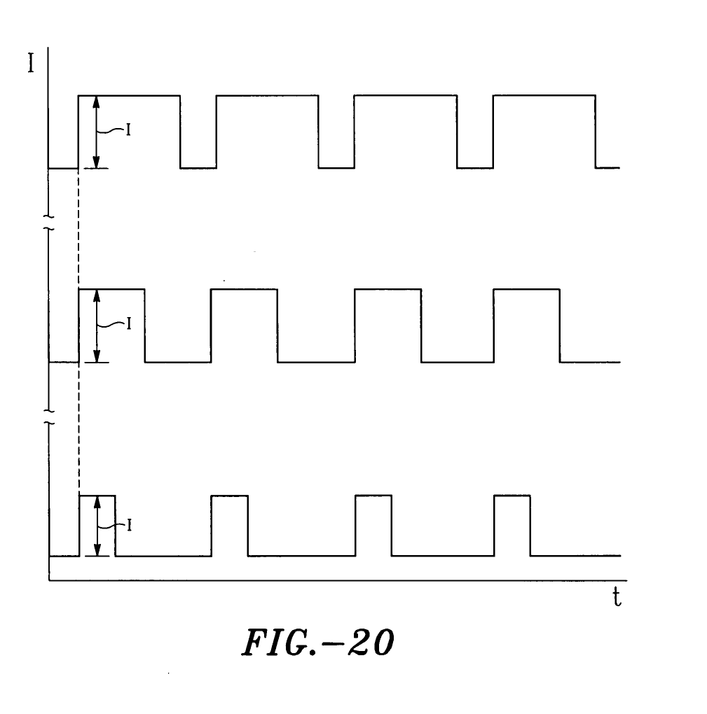

Yet another demonstration of the ability of the inventive systems and methods to maintain a therapeutic plasma concentration of Compound 1 is shown in FIG. 19. The data shown represents a comparison of in vivo electrotransport to conventional constant intravenous infusion. Specifically, Yorkshire swine were either treated with a 10 cm² electrode in communication with a hydrogel formulation of Compound 1 or with an intravenous infusion of Compound 1 at 1 mg/hr. As can be seen, the electrotransport method is capable of maintaining essentially the same level of plasma concentration as conventional intravenous infusion. This demonstrates the suitability of the inventive methods and systems for delivering precise and accurate dosages, which as discussed above, is crucial for effective anticoagulant administration.

During these studies, Primary Irritation Index (PII) data was obtained. This data is shown in Table 3, and represents standardized values of PII at the anode sites following 24-hour application of Compound 1 in hairless guinea pigs. As can be seen, the irritation both at 0.050 mA/cm² and at 0.10 mA/cm² was characterized as mild. This indicates that the electrotransport conditions suitable for maintaining a therapeutic plasma concentration of Compound I will not cause significant discomfort and can be expected to be acceptable for the patient.

TABLE 3

| | |
|---|---|
| Treatment Group | PII |
| (n = 5 per treatment group) | (Category |

| | | |
|---|---|---|
| pH 5, 0.050 | mA/cm² | 1.1 (mild) |
| pH 7, 0.050 | mA/cm² | 1.5 (mild) |
| pH 5, 0.10 | mA/cm² | 2.0 (mild) |
| pH 7, 0.10 | mA/cm² | 2.0 (mild) |

Although the above data indicates that direct current delivery at 0.050 mA/cm² and 0.10 mA/cm² electrotransport conditions do not cause significant skin irritation, alternative electrotransport conditions may be employed if desired. For example, pulsed current, alternating reverse polarity or time varying on-off current patterns may be suitable to prevent or minimize skin irritation if prolonged direct current delivery at a single location is undesirable. The electrotransport delivery devices of the invention can utilize any suitable electrical circuits in order to perform a number of functions. These complex circuits include pulsing circuits for delivering a pulsed current timing circuit for delivering agents over predetermined timing and dosing regimens, feedback regulating circuits for delivering agents in response to a sensed physical parameter, and polarity controlling circuits for periodically reversing the polarity of the electrodes. See for example, Tapper, et al., U.S. Pat. No. 4,340,047; Lattin, U.S. Pat. No. 4,456,012; Jacobsen, U.S. Pat. No. 4,141,359; and Lattin, et al., U.S. Pat. No. 4,406,658.

Thus, some embodiments of the invention can suitably utilize a pulsed (square wave) current. Duty cycle is the ratio of “on” time interval to the period of time of one cycle (i.e., the ratio of the pulse-duration time to the pulse-period) and is usually expressed as a percent. For example, if a device is “on” for 500 ms of a 1 sec cycle, then the device is operating in a 50% duty cycle. In such embodiments, the generated load current pattern makes adjustments to the load current either by changing the magnitude or by changing the duty cycle of the pulse. For example, an average current of 0-0.05 mA/cm², 10% duty cycle pulse is 0.005 mA/cm². For the purpose of this embodiment, it is stipulated that the frequency is less than 100 Hz. Doubling the preceding average current is accomplished by increasing the load current to 0-0.1 mA/cm² while keeping the duty cycle constant at 10%, or doubling the duty cycle to 20% while maintaining the load current at 0-0.05 mA/cm². (Note that these relationships are approximations.) As is well known in the art, if otherwise modulated current is used, the load current can be changed by changing the shape of the waveform. The total time of current application could also be adjusted in order to provide a desired agent delivery rate, particularly in on-demand delivery applications.

Thus, modifying the duty cycle of the pulses increases or decreases the amount of agent delivered. In this practice of the invention, the magnitude of the current pulses is selected in view of the known area of the surface from which agent is delivered, thereby defining a fixed and known current density (i.e., the ratio of current to the area from which current flows). As shown in FIG. 20 waveforms for three different pulsing electrotransport currents of the same frequency are shown having duty cycles of 75% (top waveform), 50% (middle waveform) and 25% (bottom waveform). Thus, the 25% duty cycle waveform delivers an agent transdermally by electrotransport at about one-half the dosing level of the 50% duty cycle waveform and about one-third the dosing level of the 75% duty cycle waveform.

As discussed in U.S. Pat. No. 5,983,130, which is hereby incorporated by reference in it entirety, enhanced agent delivery can be achieved by applying a current density to a body site above a critical level. Once it has been determined that a specific maximum current for a given anode surface area will provide the enhanced efficiency agent delivery discussed above, then by increasing or decreasing the duty cycle, the amount of agent delivered at the high efficiency state can be increased or decreased without causing the maximum applied current density to change. In choosing the parameters of electrotransport using this approach, the amplitude of the current pulses is selected so that the resulting current density transforms the skin into the high efficiency transfer state and the duty cycle of the current pulses is altered to adjust the agent delivery rate. Alternatively, the pulsing frequency of a pulsed current waveform is adjusted to control the overall quantity of agent delivered while maintaining current density at or above the level which transforms the skin into the high efficiency state.

Another suitable type of electrotransport delivery may be characterized as alternating reverse polarity. An example of such a system is disclosed in U.S. Pat. No. 4,406,658, which is hereby incorporated in its entirety by reference. Generally, an ionic species is used to trigger a conversion in the skin to a more permeable state, which will allow more efficient agent transfer. As is well known in the art, such a system would first drive the anionic agent counter ion from the donor reservoir and the cationic substance from the counter reservoir for the time required to convert the skin to a high efficiency state and then reverse polarity, thereby moving the agent cation into the skin.

It may be desirable to configure the electrotransport transdermal delivery device of the invention to be suited to the desired application. For example, a device configured for use in a hospital or clinic may consist of a controller or current source capable of delivering a wide array of dosing levels. As such, the hospital use system can be used to titrate the dosage to obtain and maintain the desired plasma concentration of the anticoagulant agent. Alternatively, a device configured for individual, independent use by a patient should deliver a single dose that has been determined to be therapeutically effective. Ideally, such a system should require minimal user intervention.

The systems and methods of the invention can also be used in a feedback manner to create a closed loop. Specifically, interfacing the electrotransport devices of the invention with a conventional blood clotting time monitor allows the anticoagulant agent flux to be controlled to maintain optimal anticoagulation effect. According to the invention, information from a blood clotting monitor can therefore be used to automatically adjust electrotransport conditions to vary the flux of the anticoagulant agent and thus, maintain plasma concentration of the anticoagulant at thereapeutically desired levels.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

1. A device for transdermally delivering an anticoagulant agent by electrotransport, said device comprising:
a donor reservoir having a source of said anticoagulant agent in a form to be delivered by electrotransport;
a counter reservoir;
a source of electrical power electrically connected to said reservoirs; and
a control circuit for controlling electrotransport current, said control circuit capable of effecting electrotransport conditions configured to maintain a therapeutically desired plasma concentration of said anticoagulant agent.

2. The device of claim 1, wherein said source of anticoagulant agent comprises a hydrogel formulation.

3. The device of claim 2, wherein said anticoagulant agent comprises a benzamidine derivative.

4. The device of claim 3, wherein said benzamidine derivative comprises the 2-[3-[4-(4-piperidinyloxy)anilino]-1-propenyl]benzamidine derivative shown in FIG. 3.

5. The device of claim 4, wherein said benzamidine derivative is selected from the group consisting of: N-[4-(1-acetimidoyl piperidin-4-yloxy)-3-chlorophenyl]-N-[3-(3-amidino phenyl)-2-(E)-propenyl]sulfamoyl acetic acid; N-[4-((1-acetimidoyl piperidin-4-yl)oxy)-3-carbamoyl phenyl]-N-[(E)-3-(3-amidino phenyl)-2-methyl-2-propenyl]sulfamoyl]acetic acid; N-[4-(1-aceto imidoyl piperidine-4-yloxy) phenyl]-N-[3-(3-amidino phenyl)-2-(E)-propenyl]sulfamoyl acetic acid; N-[4-(1-aceto imidoyl piperidine-4-yloxy)-3-chlorophenyl]-N-[3-(3-amidino phenyl)-2-(E)-propenyl]sulfamoyl acetic acid; N-[4-(1-aceto imidoyl piperidine-4-yloxy)-3-methylphenyl]-N-[3-(3-amidino phenyl-2-(E)-propenyl]sulfamoyl.acetic acid; N-[4-(1-aceto imidoyl piperidine-4-yloxy)-3-trifluoromethylphenyl]-N-[3-(3-amidino phenyl)-2-(E)-propenyl]sulfamoyl acetic acid; N-[4-(1-aceto imidoyl piperidine-4-yloxy)-3-carbamoylphenyl]-N-[3-(3-amidino phenyl)-2-(E)-propenyl]sulfamoyl acetic acid; N-[4-(1-acelo imidoyl piperidine-4-yloxy) phenyl]-N-[3-(3-amidino phenyl)-2-fluoro-2-(Z)-propenyl]sulfamoyl acetic acid; N-[4-(1-aceto imidoyl piperidine-4-yloxy)phenyl]-N-[3-(3-amidino phenyl)-2-methyl-2-(E)-propenyl]sulfamoyl acetic acid; and N-[4-(1-aceto imidoyl piperidine-4-yloxy)-3-carbamoylphenyl]-N-[3-(3-amidino phenyl)-2-fluoro-2-(Z)-propenyl]sulfamoyl acetic acid; and pharmacologically acceptable salts thereof.

6. The device of claim 2, wherein said anticoagulant agent comprises a naphthamidine derivative.

7. The device of claim 4, wherein said control circuit is configured to maintain said therapeutically desired plasma concentration of said anticoagulant agent in the range of approximately 20-80 ng/mL.

8. The device of claim 1, wherein said control is configured to deliver a current density in the range of approximately 0.010-0.20 mA/cm².

9. The device of claim 1, wherein said control is configured to deliver a current density in the range of approximately 0.050 mA/cm².

10. The device of claim 1, wherein said control is configured to deliver a current density in the range of approximately 0.10 mA/cm².

11. The device of claim 1, wherein said control comprises a donor electrode having an area in the range of approximately 5 to 20 cm².

12. The device of claim 4, wherein said control circuit is configured to deliver a dose of said anticoagulant agent in the range of approximately 0.5-10 mg/day.

13. The device of any of claim 12, wherein said control circuit is configured to deliver a dose of said anticoagulant agent in the range of approximately 10-50 mg/day.

14. The device of claim 13, wherein said control circuit is configured to deliver a dose of said anticoagulant agent in the range of approximately 20-40 mg/day.

15. The device of claim 1, wherein said therapeutically effective plasma concentration of said anticoagulant agent is substantially equivalent to a plasma concentration maintained by intravenous infusion.

16. The device of claim 1, wherein said control circuit is configured to deliver direct current electrotransport conditions.

17. The device of claim 1, wherein said control circuit is configured to deliver alternating reverse polarity electrotransport conditions.

18. The device of claim 1, wherein said control circuit is configured to deliver time-varying on and off electrotransport conditions.

19. The device of claim 1, further comprising a blood clotting time monitor and wherein said controller is configured to adjust time-varying electrotransport conditions in response to a signal from said blood clotting time monitor.

20. A method for maintaining a therapeutically effective plasma concentration of an anticoagulant agent; comprising the step of transdermally delivering by electrotransport an effective dose of said anticoagulant agent.

21. The method of claim 20, wherein said step of delivering said anticoagulant agent comprises delivering a benzamidine derivative.

22. The device of claim 21, wherein said step of delivering said anticoagulant agent comprises delivering a 2-[3-[4-(4-piperidinyloxy)anilino]-1-propenyl]benzamidine derivative shown in FIG. 3. ROH4746.

23. The method of claim 22, wherein said step of transdermally delivering by electrotransport an effective dose of said anticoagulant agent comprises maintaining a plasma concentration in the range of approximately 20-80 ng/mL of said benzamidine derivative shown in FIG. 3.

24. The method of claim 20, wherein said step of transdermally delivering by electrotransport an effective dose of said anticoagulant agent comprises applying a current density in the range of approximately 0.010-0.20 mA/cm².

25. The method of claim 24, wherein said step of transdermally delivering by electrotransport an effective dose of said anticoagulant agent comprises applying a current density in the range of approximately 0.050-0.10 mA/cm².

26. The method of claim 20, wherein said step of transdermally delivering by electrotransport an effective dose of said anticoagulant agent comprises delivering in the range of approximately 0.5-70 mg/day of said anticoagulant agent.

27. The method of claim 20, wherein said step of transdermally delivering by electrotransport comprises applying a pulsed current.

28. The device of claim 20, wherein said step of transdermally delivering by electrotransport comprises applying an alternating reverse polarity current.

29. The method of claim 20, wherein said step of transdermally delivering by electrotransport comprises applying a time-varying on-off current.

30. The method of claim 20, further comprising the steps of providing a blood clotting time monitor and using a signal from said blood clotting time monitor to adjust electrotransport conditions for said step of transdermally delivering by electrotransport an effective dose of said anticoagulant agent.

31. The method of claim 20, wherein said anticoagulant agent comprises a benzamidine derivative.

32. The method of claim 20, wherein said anticoagulant agent comprises a naphthamidine derivative.

33. A method for inhibiting Factor Xa in a patient comprising the step of transdermally delivering by electrotransport in the range of approximately 0.5 to 70 mg/day an anticoagulant agent.

34. The method of claim 33, wherein said anticoagulant agent comprises the 2-[3-[4-(4-piperidinyloxy)anilino]-1-propenyl]benzamidine derivative shown in FIG. 3.

35. A method of reducing risk of thromboembolic disease in a patient comprising the step of transdermally delivering by electrotransport in the range of approximately 20-40 mg/day of the 2-[3-[4-(4-piperidinyloxy)anilino]-1-propenyl]benzamidine derivative shown in FIG. 3 to maintain a plasma concentration in range of approximately 20-80 ng/mL.
